(19) United States
(12) Patent Application Publication
(10) Pub. No.: US 2026/0144772 A1
(43) Pub. Date: May 28, 2026

(54) ASPIRIN-MIMETIC ANTIOXIDANTS FOR TREATMENT OF AMYTROPHIC LATERAL SCLEROSIS

(71) Applicant: Yansong Lu, Edison, NJ (US)

(72) Inventor: Yansong Lu, Edison, NJ (US)

(21) Appl. No.: 19/019,724

(22) Filed: Jan. 14, 2025

(30) Foreign Application Priority Data
Nov. 27, 2024 (CN) 202411709739.X

(51) Int. Cl. A61K 31/265; A61K 31/4406; A61P 25/28

(52) U.S. Cl. CPC A61K 31/265; A61K 31/4406; A61P 25/28

(57) ABSTRACT

Pharmaceutical compositions comprising antioxidants as useful therapeutic agents for treatment of amyotrophic lateral sclerosis (ALS), and methods thereof, are disclosed. In particular, the present disclosure provides enteric capsules and intravenous (i.v.) drip of aspirin-mimetic antioxidant drugs in intervention of ALS.

### CROSS-REFERENCE TO RELATED APPLICATION

This application claims priority under 35 U.S.C. § 119(a) to Chinese Patent Application No. 202411709739.X, filed on Nov. 27, 2024, with the China National Intellectual Property Administration, the disclosure of which is incorporated herein by reference in its entirety for all purposes.

### Field OF THE DISCLOSURE

This disclosure relates to use of antioxidant compounds as therapeutic agents for treatment of Amyotrophic Lateral Sclerosis (ALS).

### BACKGROUND OF THE DISCLOSURE

Amyotrophic Lateral Sclerosis (ALS), also known as Lou Gehrig's disease, is a progressive neurodegenerative disease that is a fatal disease currently without effective therapy. The neurological disorder affects motor neurons in brain and spinal cord. The motor neurons are nerve cells that control voluntary muscle movement and breathing. With ALS conditions, the motor neurons are affected and degenerated, leading to paralysis and death. Inevitably, patients become paralyzed and eventually die from respiratory failures. Most people with ALS die within 3-5 years after symptoms first appear, and only ten percent survive for a decade or more. ALS is a rare disease, with two people out of a hundred-thousand population diagnosed with ALS each year, but ALS affects people of all races, ages, and genders, and the causes of ALS remain elusive. There are about thirty thousand of Americans with ALS conditions. The etiology of ALS remains unclear, although 2-5% of ALS patients are familial inherited.

There is no known cure medication, albeit four drugs had been approved by FDA for treatment of ALS. Riluzole was approved by FDA in 1995, and it can extend patients' life for 2-3 months. Edaravone was approved by FDA in 2017, and it can slow down the progression of ALS in early stage by 33.3%, compared to placebo in a 6-month clinical trial. It is worth noting that Edaravone (Radicava) failed in a clinical trial with an aim to slow down the progression of ALS in all stages.s Relyvrio (sodium phenylbutyrate/taurursodiol) was approved by FDA in 2022, and it can extend patients' life for 5.5 months when applied in early stage of ALS. Tofersen was approved by FDA in 2023, which is a mRNA drug (MW 7128 dalton) via intrathecal injection for familial-inherited ALS (genetic). The SOD1-ALS patients are accounted for 2% of total ALS patients. Tofersen reduced NfL, but did not impact the progression of ALS.

In sum, the approved drugs/therapies for ALS treatment may slow down progression of the disease, extend survival time, or improve quality of life, but they cannot reverse the progression or stop the progression of the disease, nor repair the damage of motor neurons. Therefore, effective therapeutic agents and methods remain unmet medical needs.

### SUMMARY OF THE DISCLOSURE

The present disclosure aims to meet the foregoing unmet medical needs by providing aspirin-mimetic antioxidants for treatment of ALS.

In one aspect, the present disclosure provides compounds of formula (I):

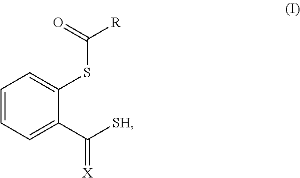

or a pharmaceutically acceptable salt thereof for use in the treatment of amyotrophic lateral sclerosis (ALS), wherein X is 0 or S, and R is alkyl or heteroaryl.

In another aspect, this disclosure provides an i.v. drip solution comprising a compound of formula (I), or a pharmaceutically acceptable salt thereof, in any embodiment disclosed, and a pharmaceutically acceptable carrier, and optionally one or more pharmaceutical excipients.

In another aspect, this disclosure provides an enteric capsule comprising a compound of formula (1), in any embodiment disclosed, and a pharmaceutically acceptable carrier, and optionally one or more pharmaceutical excipients.

In another aspect, the present disclosure provides a method of treating ALS in a subject, comprising administering to the subject a therapeutically effective amount of an i.v. drip solution comprising a compound of formula (I), or a pharmaceutically acceptable salt thereof described in any embodiment disclosed.

In another aspect, the present disclosure provides a method of treating ALS in a subject, comprising administering to the subject a therapeutically effective amount of an enteric capsule comprising a compound of formula (I), or a pharmaceutically acceptable salt thereof, described in any embodiment disclosed.

Other aspects or advantages of the present disclosure will be better appreciated in view of the following detailed description, Examples, and claims.

### DETAILED DESCRIPTION OF THE DISCLOSURE

The present disclosure provides aspirin-mimetic compounds capable of terminating free radical chain reactions, i.e., serving as free radical scavengers and/or antioxidants, useful as therapeutic agents for treatment of diseases and conditions related to reactive oxygen species (ROS) and/or reactive nitrogen species (RNS). As free radical is one of the possible key causes of many major diseases, it is plausible to find an antioxidant drug that can scavenge free radicals and show efficacy to several diseases.

In particular, this disclosure is directed to enteric capsules and/or intravenous (i.v.) drip of aspirin-mimetic antioxidants for ALS treatment. The capsules taken daily via oral could substantially reduce the progression, or even slightly reverse ALS in some cases, during a 6-month of compassionate use of the capsules requested by six ALS patients. The drug (DPL-002) showed remarkable efficacy of 67% vs Edaravone and could meet the unmet medical needs for ALS as there is currently no effective therapy for this incurable motor neuron disorder.

In one aspect, the present disclosure provides compounds of formula (I):

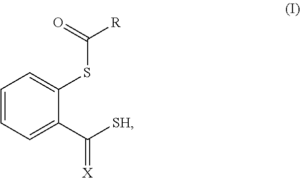

or a pharmaceutically acceptable salt thereof, for use in the treatment of amyotrophic lateral sclerosis (ALS), wherein X is O or S, and R is alkyl or heteroaryl.

In one embodiment, X is O.

In another embodiment, X is S.

In another embodiment, R is C₁₋₆ alkyl, preferably methyl or ethyl.

In another embodiment, R is a 5 to 6-membered heteroaryl, preferably pyridinyl.

In another embodiment, the compound of formula (I) is selected from:

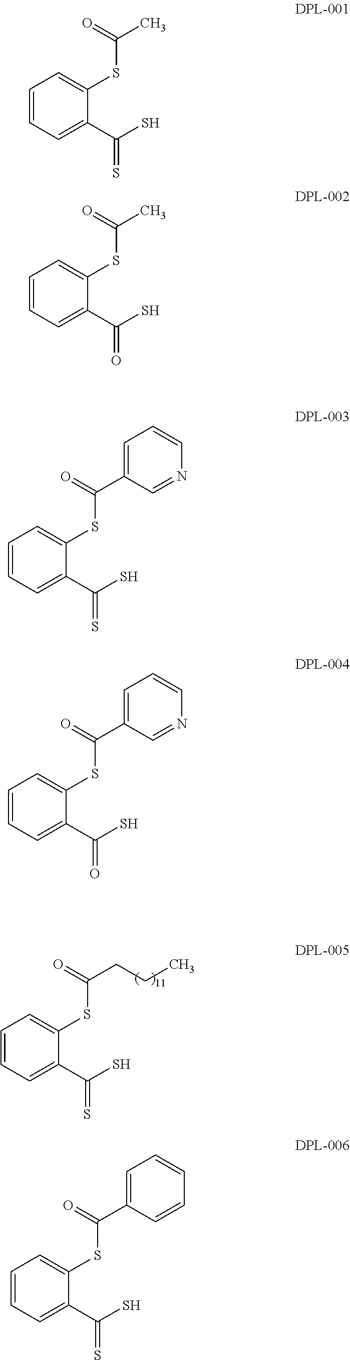

or a phannaceuically acceptable salt thereof.

In another aspect, this disclosure provides an i.v. drip solution comprising a compound of formula (I), or a pharmaceutically acceptable salt thereof, as defined above, and a pharmaceutically acceptable carrier, and optionally one or more pharmaceutical excipients.

In one embodiment of this aspect, the pharmaceutical composition is an aqueous solution for parenteral administration.

In one embodiment of this aspect, the aqueous solution is an intravenous drip.

In one embodiment of this aspect, the pharmaceutically acceptable carrier is water, and the pharmaceutical composition further comprises a pharmaceutical excipient selected from sodium chloride, potassium chloride, boric acid, sodium borate, benzalkonium chloride, glycol, glycerin, polysorbate, carboxy methylcellulose sodium, and combinations thereof.

In one embodiment of this aspect, the concentration of the API in the i.v. drip is in a range from about 0.1 mg/mL to about 30 mg/mL.

In one embodiment of this aspect, sometimes preferably, the concentration of the API in the i.v. drip is in a range from about 0.5 mg/nil, to about 30 mg/mL.

In one embodiment of this aspect, sometimes preferably, the concentration of the API in the i.v. drip is in a range from about 1 mg/mL to about 30 mg/mL.

In one embodiment of this aspect, sometimes preferably, the concentration of the API in the i.v. drip is in a range from about 0.1 mg/mL to about 20 mg/mL.

In one embodiment of this aspect, sometimes preferably, the concentration of the API in the i.v. drip is in a range from about 0.5 mg/mL to about 20 mg/mL.

In one embodiment of this aspect, sometimes preferably, the concentration of the API in the i.v. drip is in a range from about 1 mg/mL to about 20 mg/mL.

In one embodiment of this aspect, sometimes preferably, the concentration of the API in the i.v. drip is in a range from about 0.1 mg/mL to about 10 mg/mL.

In one embodiment of this aspect, sometimes preferably, the concentration of the API in the i.v. drip is in a range from about 0.5 mg/mL to about 10 mg/mL.

In one embodiment of this aspect, sometimes preferably, the concentration of the API in the i.v. drip is in a range from about 1 mg/mL to about 10 mg/mL.

In another aspect, the present disclosure provides a method of treating amyotrophic lateral sclerosis (ALS), comprising administering to a subject in need of treatment a therapeutically effective amount of an active pharmaceutical ingredient (API) selected from:

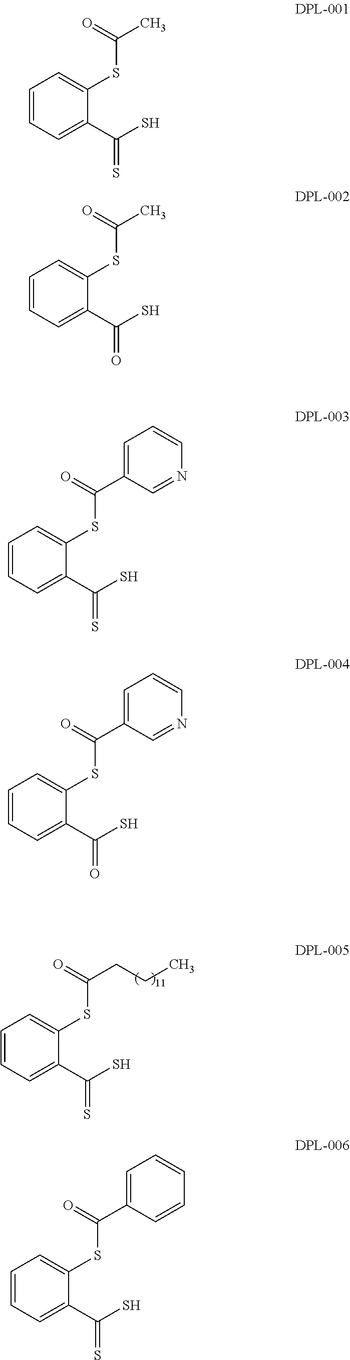

or a pharmaceutically acceptable salt thereof.

In some, embodiments of this aspect, sometimes preferably, the active pharmaceutical ingredient is DPL-002 or DPL-004.

In some embodiments of this aspect, sometimes more preferably, the active pharmaceutical ingredient is selected from DPL-002.

In another aspect, the present disclosure provides an intravenous (i.v.) drip solution comprising an active pharmaceutical ingredient (API) compound selected from DPL-001, DPL-002, DPL-003, DPL-004, DPL-005, and DPL-006, a pharmaceutically acceptable carrier, and optionally one or more pharmaceutical excipients.

In some embodiments of this aspect, sometimes preferably, the active pharmaceutical ingredient (API) is DPL-002 or DPL-004.

In some embodiments of this aspect, sometimes more preferably, the active pharmaceutical ingredient is DPL-002.

In some embodiments of this aspect, sometimes preferably, the pharmaceutically acceptable carrier is water, and the i.v. drip solution further comprises a pharmaceutical excipient independently selected from sodium chloride, potassium chloride, sodium hydrosulfite, and combinations thereof.

In some embodiments of this aspect, sometimes preferably, the concentration of the API in the i.v. drip solution is in a range from about 0.1 mg/mL to about 30 mg/m.

In one embodiment of this aspect, sometimes preferably, the concentration of the API in the i.v. drip is in a range from about 0.5 mg/mL to about 30 mg/mL.

In one embodiment of this aspect, sometimes preferably, the concentration of the API in the i.v. drip is in a range from about 1 mg/mL to about 30 mg/mL.

In one embodiment of this aspect, sometimes preferably, the concentration of the API in the i.v. drip is in a range from about 0.1 mg/mL to about 20 mg/mL.

In one embodiment of this aspect, sometimes preferably, the concentration of the API in the i.v. drip is in a range from about 0.5 mg/mL to about 2.0 mg/mL.

In one embodiment of this aspect, sometimes preferably, the concentration of the API in the i.v. drip is in a range from about 1 mg/mL to about 20 mg/mL.

In one embodiment of this aspect, sometimes preferably, the concentration of the API in the i.v. drip is in a range from about 0.1 mg/mL to about 10 mg/mL.

In one embodiment of this aspect, sometimes preferably, the concentration of the API in the i.v. drip is in a range from about 0.5 mg/mL to about 10 mg/mL.

In some embodiments of this aspect, sometimes preferably, the concentration of the API in the i.v. drip is in a range from about 1 mg/mL to about 10 mg/mL.

In some embodiments of this aspect, the i.v. drip solution has a pH in the range from 6 to 8, inclusive.

In some embodiments of this aspect, sometimes preferably, the i.v. drip solution has a pH in the range from 6.5 to 7.5, inclusive.

In some embodiments of this aspect, sometimes preferably, the i.v. drip solution has a pH in the range from 6.8 to 7.8, inclusive.

In some embodiments of this aspect, sometimes preferably, the i.v. drip solution has a pH at 7.0, inclusive.

In some embodiments of this aspect, sometimes preferably, the pH of the i.v. drip solution is adjusted using an acid selected from hydrochloric acid, sulfuric acid, phosphoric acid, and acetic acid; and/or a base selected from sodium hydroxide, potassium hydroxide, lithium hydroxide, and ammonium hydroxide; and/or a sodium, potassium, or lithium salt of carbonate, bicarbonate, phosphate, hydrogen phosphate sodium, dihydrogen phosphate, acetate, and the like.

In another aspect, the present disclosure provides an enteric capsule comprising an active pharmaceutical ingredient (API) compound selected from DPL-001, DPL-002, DPL-003, DPL-004, DPL-005, and DPL-006, and optionally one or more pharmaceutical excipients.

In some embodiments of this aspect, sometimes preferably, the active pharmaceutical ingredient is DPL-002 or DPL-004.

In some embodiments of this aspect, sometimes more preferably, the active pharmaceutical ingredient is DPL-002.

In some embodiments of this aspect, sometimes preferably, the pharmaceutical excipient is independently selected from starch powder, purified water, NaCl, KCl, dicalcium phosphate, magnesium stearate, carboxy methylcellulose sodium, and combinations thereof.

In another aspect, the present disclosure provides a method of treating ALS in a subject, comprising administering to the subject a therapeutically effective amount of the i.v. drip solution according to any embodiments disclosed.

In some embodiments of this aspect, sometimes preferably, the dose range of API in the i.v. drip solution administered to the subject is from about 5 mg to about 1500 mg daily.

In some embodiments of this aspect, the dose range of API in the i.v. drip solution administered to the subject is from about 0.1 mg/kg body weight to about 30 mg/kg body weight.

In some embodiments of this aspect, sometimes preferably, the dose range of API in the i.v. drip solution administered to the subject is from about 0.5 mg/kg body weight to about 30 mg/kg body weight.

In some embodiments of this aspect, sometimes preferably, the dose range of API in the i.v. drip solution administered to the subject is from about 1 mg/kg body weight to about 30 mg/kg body weight.

In some embodiments of this aspect, sometimes preferably, the dose range of API in the i.v. drip solution administered to the subject is from about 0.1 mg/kg body weight to about 20 mg/kg body weight.

In some embodiments of this aspect, sometimes preferably, the dose range of API in the i.v. drip solution administered to the subject is from about 0.5 mg/kg body weight to about 20 mg/kg body weight.

In some embodiments of this aspect, sometimes preferably, the dose range of API in the i.v. drip solution administered to the subject is from about 1 mg/kg body weight to about 20 mg/kg body weight.

In some embodiments of this aspect, sometimes preferably, the dose range of API in the i.v. drip solution administered to the subject is from about 0.1 mg/kg body weight to about 10 mg/kg body weight.

In some embodiments of this aspect, sometimes preferably, the dose range of API in the i.v. drip solution administered to the subject is from about 0.5 mg/kg body weight to about 10 mg/kg body weight.

In some embodiments of this aspect, sometimes preferably, the dose range of API in the i.v. drip solution administered to the subject is from about 1 mg/kg body weight to about 10 mg/kg body weight.

In some embodiments of this aspect, sometimes preferably, the dose range of API in the i.v. drip solution administered to the subject is from about 0.5 μmol/kg body weight to about 150 μmol/kg body weight.

In some embodiments of this aspect, sometimes preferably, the dose range of API in the i.v. drip solution administered to the subject is from about 0.5 μmol/kg body weight to about 100 μmol/kg body weight.

In some embodiments of this aspect, sometimes preferably, the dose range of API in the i.v. drip solution administered to the subject is from about 0.5 μmol/kg body weight to about 50 μmol/kg body weight.

In some embodiments of this aspect, sometimes preferably, the dose range of API in the i.v. drip solution administered to the subject is from about 1.0 μmol/kg body weight to about 150 μmol/kg body weight.

In some embodiments of this aspect, sometimes preferably, the dose range of API in the i.v. drip solution administered to the subject is from about 1.0 μmol/kg body weight to about 100 μmol/kg body weight.

In some embodiments of this aspect, sometimes preferably, the dose range of API in the i.v. drip solution administered to the subject is from about 1.0 μmol/kg body weight to about 50 μmol/kg body weight.

In some embodiments of this aspect, sometimes preferably, the dose range of API in the i.v. drip solution administered to the subject is from about 2.0 μmol/kg body weight to about 150 μmol/kg body weight.

In some embodiments of this aspect, sometimes preferably, the dose range of API in the i.v. drip solution administered to the subject is from about 2.0 μmol/kg body weight to about 100 μmol/kg body weight.

In some embodiments of this aspect, sometimes preferably, the dose range of API in the i.v. drip solution administered to the subject is from about 2.0 μmol/kg body weight to about 50 μmol/kg body weight.

In sone embodiments of this aspect, sometimes preferably, the dose range of API in the i.v. drip solution administered to the subject is from about 5.0 gmol/kg body weight to about 150 μmol/kg body weight.

In some embodiments of this aspect, sometimes preferably, the dose range of API in the i.v. drip solution administered to the subject is from about 5.0 μmol/kg body weight to about 100 μmol/kg body weight.

In some embodiments of this aspect, sometimes preferably, the dose range of API in the i.v. drip solution administered to the subject is from about 5.0 μmol/kg body weight to about 50 μmol/kg body weight.

In some embodiments of this aspect, sometimes preferably, the i.v. drip solution is administered to the subject in a frequency from one time daily to 5 times daily.

In some embodiments of this aspect, sometimes preferably, the i.v. drip solution is administered to the subject in a frequency from one time daily to 3 times daily.

In some embodiments of this aspect, sometimes preferably, the i.v. drip solution is administered to the subject in a frequency from one to two times daily.

In some embodiments of this aspect, sometimes preferably, the i.v. drip solution is administered to the subject in a frequency of once daily.

In any of the aforementioned embodiments, the API is sometimes preferably DPL-002 or DPL-004, and sometimes more preferably DPL-002.

In another aspect, the present disclosure provides a method of treating ALS in a subject, comprising administering to the subject a therapeutically effective amount of the enteric capsule according to any embodiments disclosed.

In some embodiments of this aspect, sometimes preferably, the dose of the API in the enteric capsule is in a range from about 10 mg to about 3000 mg daily.

In some embodiments of this aspect, sometimes preferably, the dose of the API in the enteric capsule is in a range from about 0.2 mg/kg body weight to about 60 mg/kg body weight.

In some embodiments of this aspect, sometimes preferably, the dose of the API in the enteric capsule is in a range from about 0.2 mg/kg body weight to about 40 mg/kg body weight.

In some embodiments of this aspect, sometimes preferably, the dose of the API in the enteric capsule is in a range from about 0.2 mg/kg body weight to about 20 mg/kg body weight.

In some embodiments of this aspect, sometimes preferably, the dose of the API in the enteric capsule is in a range from about 0.2 mg/kg body weight to about 10 mg/kg body weight.

In some embodiments of this aspect, sometimes preferably, the dose of the API in the enteric capsule is in a range from about 0.4 mg/kg body weight to about 60 mg/kg body weight.

In some embodiments of this aspect, sometimes preferably, the dose of the API in the enteric capsule is in a range from about 0.4 mg/kg body weight to about 40 mg/kg body weight.

In some embodiments of this aspect, sometimes preferably, the dose of the API in the enteric capsule is in a range from about 0.4 mg/kg body weight to about 20 mg/kg body weight.

In some embodiments of this aspect, sometimes preferably, the dose of the API in the enteric capsule is in a range from about 0.4 mg/kg body weight to about 10 mg/kg body weight.

In some embodiments of this aspect, sometimes preferably, the dose of the API in the enteric capsule is in a range from about 0.6 ng/kg body weight to about 60 ng/kg body weight.

In some embodiments of this aspect, sometimes preferably, the dose of the API in the enteric capsule is in a range from about 0.6 mg/kg body weight to about 40 mg/kg body weight.

In some embodiments of this aspect, sometimes preferably, the dose of the API in the enteric capsule is in a range from about 0.6 mg/kg body weight to about 20 mg/kg body weight.

In some embodiments of this aspect, sometimes preferably, the dose of the API in the enteric capsule is in a range from about 0.6 mg/kg body weight to about 10 mg/kg body weight.

In some embodiments of this aspect, sometimes preferably, the dose of the API in the enteric capsule is in a range from about 0.8 mg/kg body weight to about 60 mg/kg body weight.

In some embodiments of this aspect, sometimes preferably, the dose of the API in the enteric capsule is in a range from about 0.8 mg/kg body weight to about 40 mg/kg body weight.

In some embodiments of this aspect, sometimes preferably, the dose of the API in the enteric capsule is in a range from about 0.8 mg/kg body weight to about 20 mg/kg body weight.

In some embodiments of this aspect, sometimes preferably, the dose of the API in the enteric capsule is in a range from about 0.8 mg/kg body weight to about 10 mg/kg body weight.

In some embodiments of this aspect, sometimes preferably, the dose of the API in the enteric capsule is in a range from about 1.0 mg/kg body weight to about 60 mg/kg body weight.

In some embodiments of this aspect, sometimes preferably, the dose of the API in the enteric capsule is in a range from about 1.0 mg/kg body weight to about 40 mg/kg body weight.

In some embodiments of this aspect, sometimes preferably, the dose of the API in the enteric capsule is in a range from about 1.0 mg/kg body weight to about 20 mg/kg body weight.

In some embodiments of this aspect, sometimes preferably, the dose of the API in the enteric capsule is in a range from about 1.0 mg/kg body weight to about 10 mg/kg body weight.

In some embodiments of this aspect, sometimes preferably, the dose of the API in the enteric capsule is in a range from about 1 μmol/kg body weight to about 300 μmol/kg body weight.

In some embodiments of this aspect, sometimes preferably, the dose of the API in the enteric capsule is in a range from about 1 μmol/kg body weight to about 300 μmol/kg body weight.

In some embodiments of this aspect, sometimes preferably, the dose of the API in the enteric capsule is in a range from about 1 μmol/kg body weight to about 200 μmol/kg body weight.

In some embodiments of this aspect, sometimes preferably, the dose of the API in the enteric capsule is in a range from about 1 μmol/kg body weight to about 100 μmol/kg body weight.

In some embodiments of this aspect, sometimes preferably, the dose of the API in the enteric capsule is in a range from about 1 μmol/kg body weight to about 50 μmol/kg body weight.

In some embodiments of this aspect, sometimes preferably, the dose of the API in the enteric capsule is in a range from about 2 μmol/kg body weight to about 300 μmol/kg body weight.

In some embodiments of this aspect, sometimes preferably, the dose of the API in the enteric capsule is in a range from about 2 μmol/kg body weight to about 200 μmol/kg body weight.

In some embodiments of this aspect, sometimes preferably, the dose of the API in the enteric capsule is in a range from about 2 gmol/kg body weight to about 100 μmol/kg body weight.

In some embodiments of this aspect, sometimes preferably, the dose of the API in the enteric capsule is in a range from about 2 μmol/kg body weight to about 50 μmol/kg body weight.

In some embodiments of this aspect, sometimes preferably, the dose of the API in the enteric capsule is in a range from about 5 μmol/kg body weight to about 300 μmol/kg body weight.

In some embodiments of this aspect, sometimes preferably, the dose of the API in the enteric capsule is in a range from about 5 μmol/kg body weight to about 200 μmol/kg body weight.

In some embodiments of this aspect, sometimes preferably, the dose of the API in the enteric capsule is in a range from about 5 μmol/kg body weight to about 100 μmol/kg body weight.

In some embodiments of this aspect, sometimes preferably, the dose of the API in the enteric capsule is in a range from about 5 μmol/kg body weight to about 50 μmol/kg body weight.

In some embodiments of this aspect, sometimes preferably, the dose of the API in the enteric capsule is in a range from about 10 μmol/kg body weight to about 300 μmol/kg body weight.

In some embodiments of this aspect, sometimes preferably, the dose of the API in the enteric capsule is in a range from about 10 μmol/kg body weight to about 200 gmol/kg body weight.

In some embodiments of this aspect, sometimes preferably, the dose of the API in the enteric capsule is in a range from about 10 μmol/kg body weight to about 100 gmol/kg body weight.

In some embodiments of this aspect, sometimes preferably, the dose of the API in the enteric capsule is in a range from about 10 μmol/kg body weight to about 50 μmol/kg body weight.

In some embodiments of this aspect, sometimes preferably, the enteric capsule is administered to the subject in a frequency from 1 time daily to 5 times daily.

In some embodiments of this aspect, sometimes preferably, the enteric capsule is administered to the subject in a frequency from 1 time daily to 3 times daily.

In some embodiments of this aspect, sometimes preferably, the enteric capsule is administered to the subject in a frequency from 1 time daily to 2 times daily.

In some embodiments of this aspect, sometimes preferably, the enteric capsule is administered to the subject in a frequency of once daily.

In any of the aforementioned embodiments, the API is sometimes preferably DPL-002 or DPL-004, and sometimes more preferably DPL-002.

In another aspect, the present disclosure is directed to use of an aspirin-mimetic antioxidant of formula (I) according to any embodiment disclosed herein in the manufacture of a medicament for treatment of ALS in a subject.

In some embodiments, the aspirin-mimetic antioxidant is selected from DPL-001, DPL-002, DPL-003, DPL-004, DPL-005, and DPL-006.

In some embodiments, the medicament is an aforementioned i.v. drip solution.

In some embodiments, the medicament is an aforementioned enteric capsule.

In one embodiment, the subject is a human.

As would be understood by a person of ordinary skill in the art, the present disclosure encompasses any reasonable combinations of the embodiments disclosed.

So far, all known antioxidants used in preclinical and clinical studies are not good terminators of free radicals. When those antioxidants quench the primary free radicals, they themselves become new free radicals, called secondary free radicals. The secondary free radicals are less reactive compared to the primary free radicals. With a lower reactivity, the secondary free radicals can live longer and thus can travel a longer distance within cell/biological system compared to the corresponding primary free radicals. Based on the molecular structures of antioxidants, some secondary free radicals derived from the antioxidants, though less reactive, could reach some pivotal parts and harm DNA, lipids, and proteins via free radical oxidation. There are two explanations. First, although they are less reactive compared to the primary free radicals, the secondary free radicals are still strong enough to directly oxidize some vulnerable DNA, lipids, and proteins, etc. Second, the secondary free radicals may be too weak to directly oxidize biomolecules such as DNA, lipids, and proteins, but with some metal cation (Fe, Cu, etc.) present as the catalyst for electron transfer in vivo, they can readily oxidize some DNA, lipids, and proteins, etc.

For example, vitamin E is a strong antioxidant. However, when it quenches a free radical, it becomes a free radical itself, called the secondary free radical in the propagation process. The free radical form of vitamin E is a pro-oxidant, as it can still oxidize some other molecules by taking in an electron or hydrogen radical. Thus, the free radical form of vitamin E can still harn, especially in the presence of some metal cation (Fe, Cu, etc.) as the catalyst for electron transfer. Therefore, vitamin E is not a good scavenger of free radicals in this regard. The same conclusion can be drawn for various other known antioxidants.

Free radical scavengers as antioxidant drugs would effectively terminate free radicals, to avoid the lengthy propagation of free radicals and to reduce the life expectancy of free radicals in vivo. This logic prompts innovative designs of aspirin-mimics in this disclosure. Preclinical efficacy tests on the aspirin-mimetic antioxidants of this disclosure have reversed cataract in the rat model, suppressing the release of various cytokines on cell-based assay in anti-inflammation. See U.S. Pat. No. 11,826,355, which is hereby incorporated by reference in its entirety for all purposes.

The chemical activity of free radicals can usually be completely quenched via termination. Without intending to be bound by theory, termination of free radicals is believed to be a key working mechanism of the aspirin-mimetics of this disclosure. A simple and effective method to terminate free radicals is to let one free radical react with another free radical to form a coupling compound. Disulfide has the highest bond energy among all interested functional groups (N—N, O—O, S—S, Se—Se) and thiol is hence one of the most powerful scavengers of free radicals among them. See, e.g., U.S. Pat. No. 9,994,519, which is hereby incorporated by reference in its entirety for all purposes.

It is well known that the antioxidant strength of thiophenol is much stronger than that of alkyl thiol like cysteine and ALA (alpha lipoic acid). Thus, thiophenol is the focus of designing aspirin-mimetic antioxidants in this disclosure. Based on chemical kinetics, a dithiol-containing molecule can form a 5-membered ring (via intramolecular disulfide bond) faster than to form an intermolecular disulfide bond (non-cyclic chain in general). A disulfide formed in a 5-membered ring is also stabler than that formed in open chain. Plus, a 5-membered ring disulfide compound as 2b herein is formed with much more favored bond angles than that of ALA. Thus, the aspirin-mimetics in this disclosure are much better free radical scavengers than ALA.

Thio-aspirin-mimetics can efficiently terminate free radicals. The mechanism of how they effectively terminate free radicals is illustrated in Scheme 1.

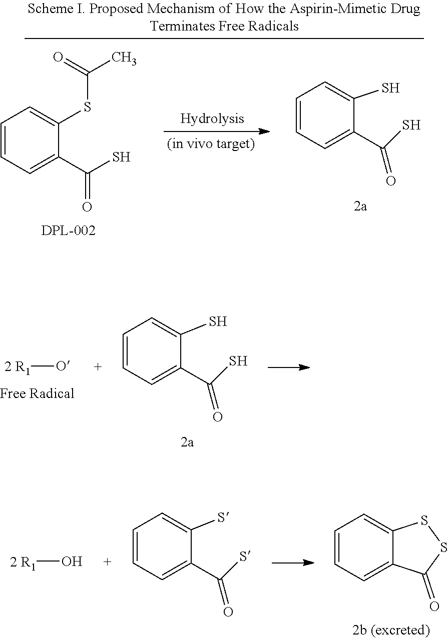

The “druggability” of aspirin is utilized in the design of aspirin-mimetic antioxidants in this disclosure. To inherit the “druggability” from aspirin, the aspirin-mimetic antioxidants will keep the essential skeleton of aspirin molecule, but with sulfur atom to replace oxygen atom in a pair of key functional groups. In the periodic table of the elements, sulfur and oxygen are in the same “oxygen group” and are of similar properties. The replacement of oxygen with sulfur will avoid any “abrupt change” in molecular structure and the aspirin-mimetic antioxidants thus constructed would still keep the “druggability” of aspirin. In addition, the replacement of oxygen with sulfur will enable the molecules to be the strongest possible scavenger of free radicals, as discussed above. Plus, it will enable the molecules to participate in “thiol-disulfide exchange” (Scheme II) and could reverse some miss-folded proteins caused by aberrant disulfide crosslinking. Based on SHAB, the replacement of oxygen with sulfur will enable the molecules to form a stronger chelate with heavy metals (Scheme III), which is because sulfide or thiolate is a softer base and has higher affinity to heavy metal cations (soft acids) like mercury, cadmium, lead, iron, copper, etc. Hence, the aspirin-mimetics in this project could efficiently remove heavy metals out of the body. The chelation power is subtle, as too weak like aspirin or too strong like EDTA has proven no good. The active ingredients of the aspirin-mimetics, like 2a (Scheme III), may fit in as a perfect one.

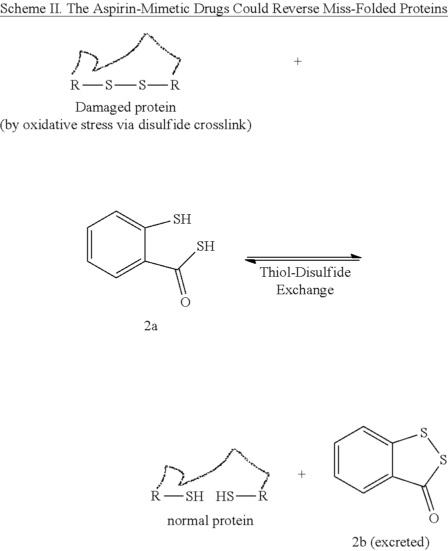

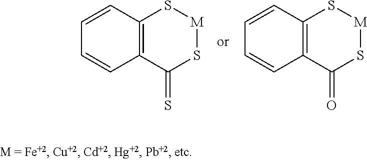

Scheme III. The Aspirin-Mimetic Drugs can Selectively Form Stable Chelate with Heavy Metals

Antioxidant “drugs” in this disclosure should be in their “reduced forms”. The disulfides are “oxidized form” that is not active and has no more capability of quenching free radicals. Thiol and thiophenol are the “reduced forms” of antioxidants, but they are unstable for storage because they are prone to air-oxidation. Hence, thiophenol group in this disclosure must be chemically protected by other functional groups. At this point, the protected one is a prodrug that can release the drug as active “reduced form” in vivo where the protection group is chemically disconnected. The protection groups selected in this disclosure also act as penetration enhancer, efficacy enhancer, selectivity enhancer, or a combination among them. In addition, the protection groups must have no toxicity issue.

The aspirin-mimetics in this disclosure are summarized in several models of molecular structure. Compounds DPL-001 and DPL-002 are the simplest forms of novel aspirin-mimetics in this disclosure. Compound 1a, the mimetic of salicylic acid, would be the active form of compound DPL-001 as free radical scavenger in vivo. Compound 2a, the mimetic of salicylic acid, would be the active form of compound DPL-002 as free radical scavenger in vivo Compounds 1b and 2b are known, but chemically prepared via different approaches. Aspirin-mimetics in this disclosure are expected being hydrolyzed in vivo, giving compound 2a or 1a.

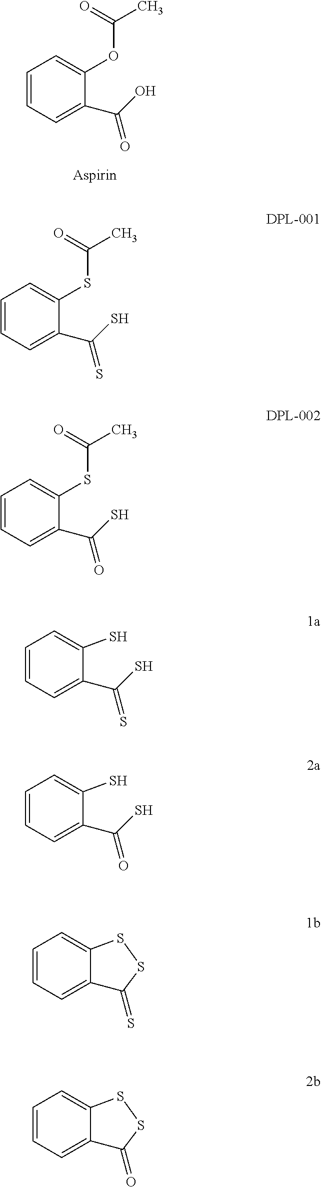

The mechanism of how a typical novel aspirin-mimetic works is proposed in Scheme 1 and Scheme IV. The compound DPL-004 is delivered to the target cell such as neuron, where it is hydrolyzed to 2a. Compound 2a can efficiently terminate two free radicals in vivo. Thus, the dose of the aspirin-mimetic drugs can be halved in this regard and so does the cost. More importantly, this effective termination of free radicals would minimize any harm from the primary free radicals and the secondary free radicals so forth yielded in the propagation chain down the track.

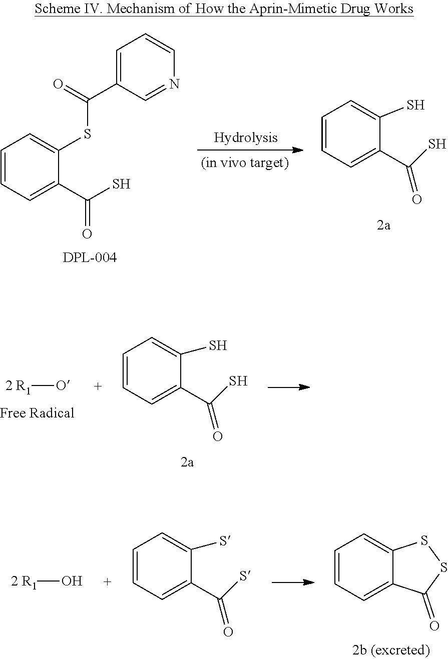

In sum, each aspirin-mimetic molecule in this disclosure consists of at least a pair of two functional groups. When oxidized, the configuration allows it to form a 5-membered ring via an intramolecular disulfide bond. The pair of functional groups consists of one thiol (—SH); and one carbodithioic acid (—CSSH), or one carbothioic acid (—COSH). Each of the pair of functional groups is covalently single-bonded with a carbon atom located on aromatic ring, or on substituted aromatic ring. The aromatic ring can be either regular aromatic ring or aromatic heterocyclic ring, with any combinations among them.

Each of the pair of functional groups can be covalently single-bonded with other functional groups selected from acetyl (—Ac), amino acid residue, vitamin B's residue, choline, dopamine, carbohydrate, nucleic base, citric acid, succinic acid, heterocycles, etc. These functional groups would act as protection group, penetration enhancer, efficacy enhancer, selectivity enhancer, or a combination among them.

As free radical is one of the possible key causes of many major diseases, it is plausible to find an antioxidant drug that can scavenge free radicals and show efficacy to many diseases like Alzheimer's disease (AD), Parkinson's disease (PD), Huntington's disease (HD), amyotrophic lateral sclerosis (ALS), diabetes, cancers, atherosclerosis, cardiovascular disease, renal disease, hypertension, hyperlipidemia, rheumatoid arthritis, lupus, multiple sclerosis (MS), gout, inflammation, pain, acne, aging, stroke, depression, cataract, glaucoma, age-related macular degeneration and HIV, and so forth. The novel antioxidants in this disclosure could bring answer to major diseases like ALS.

Unless defined otherwise, all terms used herein have the same meaning as is commonly understood by one of ordinary skill in the art to which this disclosure belongs. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural reference, and vice versa. Any plural forms include singular reference, unless the context clearly dictates otherwise.

The term “about” or “approximately”, unless otherwise defined, generally includes up to plus or minus 10% of the indicated number. For example, “about 10%” may indicate a range of 9% to 11%, and “about 20” may mean from 18 to 22. Sometimes preferably, “about” includes up to plus or minus 5% of the indicated value. Alternatively, “about” includes up to plus or minus 5% of the indicated value. When “about” is used before a range, it is applicable to both the lower end and the upper end of a range.

The term “substantially” as herein used means “for the most part” or “essentially,” as would be understood by a person of ordinary skill in the art, and if measurable quantitatively, refers to at least 90%, preferably at least 95%, more preferably at least 98%.

The terms “comprising”, “having”, “including”, and “containing”, or the like, are to be construed as open-ended terms (i.e., meaning “including, but not limited to”) unless otherwise noted.

The term “effective amount” refers to the amount necessary or sufficient to realize a desired biologic or therapeutic effect.

The term “therapeutically effective amount” means an amount effective to deliver a therapeutically effective amount of an amount of active agent needed to delay the onset of, inhibit the progression of, or halt altogether the particular disease, disorder or condition being treated, or to otherwise provide the desired effect on the subject to be treated. As one of ordinary skill in the art would understand, a therapeutically effective amount varies with the patient's age, condition, and gender, as well as the nature and extent of the disease, disorder or condition in the patient, and the dosage may be adjusted by the individual physician (or veterinarian).

The term “pharmaceutically acceptable” describes a material that is not biologically or otherwise undesirable, i.e., without causing an unacceptable level of undesirable biological effects or interacting in a deleterious manner.

The term “pharmaceutically acceptable salt” refers to a salt of the compound disclosed herein, which may be selected from the group consisting of inorganic and organic salts. The salts are safe and effective for use in the body of a mammal and possess the requisite biological activity. The salts may be prepared separately during the final separation and purification of the compound, or by reacting an appropriate group with an appropriate base or acid. Bases commonly used to form pharmaceutically acceptable salts include inorganic bases such as sodium hydroxide and potassium hydroxide, and organic bases such as amine. Acids commonly used to form pharmaceutically acceptable salts include inorganic acids and organic acids.

The terms “treating” and “treatment”, or the like, refer to reversing, alleviating, inhibiting, or slowing the progress of the disease, disorder, or condition to which such terms apply, or one or more symptoms of such disease, disorder, or condition.

The term “subject” or “patient” used herein refers to a human patient.

The following non-limiting examples will further illustrate certain aspects of the present disclosure.

### EXAMPLES

### Chemical Synthesis of the Aspirin-Mimetics

According to the procedure reported in J. Am. Chem. Soc., 1989. 111, 654-658, lithiation of thiophenol gave ortho-directed lithiation intermediate, a di-lithium salt species. That di-lithium salt species was allowed to react with carbon disulfide, followed by acetylation (via acetic anhydride), to afford compound 1 (DPL-001) in a good yield (Scheme V). Similarly, the di-lithiunm salt species can react with carbonyl sulfide, followed by acetylation (via acetic anhydride), to afford compound 2 (DPL-002) (Scheme V). Other aspirin-mimetic compounds were obtained when corresponding acylation reagents were applied.

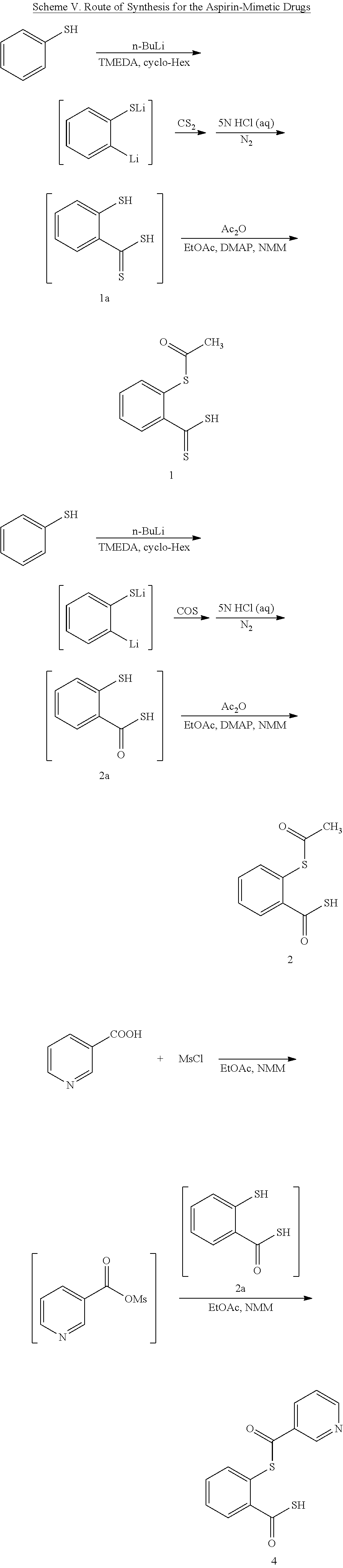

DPL-001: ¹H-NMR (400 MHz, CD₂Cl₂) δ (ppm) 7.443 (s, 4H), 2.421 (d, J=1.6 Hz, 3H); ¹³C-NMR (100 MHz, CD₂Cl₂) δ193.850, 134.538, 129.371, 129.113, 129.083, 128.210, 127.512, 127239, 29.989.

DPL-002: ¹H-NMR (400 MHz, CDCl₃) δ (ppm) 8.0-8.1-5 (m, 1H), 7.55-7.65 (m, 2H), 7.45-7.55 (m, 1H), 2.46 (s, 3H); ¹³CNMR (100 MHz, CDCl₃) δ193.149, 171.653, 136.758, 132.821, 132.580, 131.866, 129.408, 129.385, 30.365.

DPL-003: ¹H-NMR (400 MHz, DMSO-d₆) δ (ppm) 9.148 (d, J=2.0 Hz, 1H), 8.936 (dd, 1=1.6, 5.2 Hz, 1H), 8.40-8.60 (m, 1H), 7.70-7.90 (m, 1H), 7.40-7.60 (m, 5H); ¹³C—NIR (100 MHz, DMSO-d₆) δ186.322, 150.983, 146.113, 144.557, 135.446, 133.420, 130.750, 128.625, 128.208, 128.109, 124.802, 124.240, 123.739; MS(m/z) 292 (M+H)⁺, 248 (M+H−CS, 100)⁺.

DPL-004: ¹H-NMR (500 MHz, CDCl₃) δ (ppm) 9.179 (d, J=2.0 Hz, 1H), 8.759 (dd, J=: 2.0, 5.0 Hz, 1H), 8.07 (dt, J=2.0, 8.0 Hz, 1H), 7.35-7.48 (m, 6H); ¹³C-NMR (125 MHz, CDCl₃) δ188.837, 153.951, 148.590, 135.022(2), 134.828, 132.412, 129.917(2), 129.438(2), 126.291, 123.738; MS(m/z) 274 (M−H)⁺, 216 (MH+H−OCS, 100)⁺.

DPL-005: ¹H-NMR (500 MHz, CDCl₃): δ (ppm) 7.468 (s, 4H). 2.713 (t, J=2.5 Hz, 2H), 1.772 (quin, J=2.5 Hz, 2H), 1.30-1.50 (m, 20H), 0.948 (t, J=6.6 Hz, 3 Hz); C-NMR (125 MHz, CDC₃): δ 198.574, 135.476, 130.264, 130,135, 130.059, 128.989, 128.519, 128.147, 44.739, 32.927, 30.673, 30.643 (2C), 30.582, 30.415, 30.355, 30.248, 29.968, 26.607, 23.694, 15.121.

DPL-006: ¹H-NMR (400 MHz, CDCl₃): δ (ppm) 8.01-8.07 (i, 2H), 7.58-7.65 (m, 1H), 7.40-7.60 (m, 7H); ¹³C-NMR (100 MHz, CDCl₃): δ 190.170, 136.632, 135.114, 134.470, 133.680, 129.545, 129.462, 129.272, 129.227, 128.764 (2C), 127.497 (2C), 127.337.

A synthetic route for preparation of DPL-002 was shown above. The crystalline of DPL-002 was thus obtained in purity over 97%. The enteric capsules were filled with DPL-002 accordingly. No excipients were needed to prepare for the capsules.

### Compassionate Use of Capsule (DPI-002)

1. One capsule daily along with warm water 1 h before breakfast.

2. The capsules were stored in refrigerator (2-8° C.) before use.

3. The dose climbing process: placebo for one week; 10 mg daily for 10 days; 20 mg daily for 5 days; 40 mg daily for 15 days; 50 mg daily for 10 days; 100 mg daily for 58 days; 150 mg daily for 35 days; 200 mg daily for 25 days, and 100 mg daily for 20 days.

4. ALSFRS-R scores were acquired monthly.

TABLE 1
ALSFRS-R Score Before and After

| | | | | ALSFRS-R |
|---|---|---|---|---|
| Patient | Gender/ | ALS | ALSFRS-R Score | Score |
| # | Age | Stage | 0 and 6^{th} month | Change |
| 1 | F/54 | Early | 40 (0), 38 (6^{th} month) | −2 |
| 2 | F/49 | Early | 46 (0 month), 46 (6^{th} month) | 0 |
| 3 | F/52 | Middle | 34 (0 month), 26 (6^{th} month) | −8 |
| 4 | F/60 | Middle | 32 (0 month), 32 (6^{th} month) | 0 |
| 5 | M/39 | Late | 22 (0 month), 23 (6^{th} month) | 1 |
| 6 | M/47 | Late | 23 (0 month), 22 (6^{th} month) | −1 |

### 5. Data Process

ALSFR S-R score is an important indicator to measure the condition of ALS patients, and the change of this indicator before and after use of a drug can reflect the efficacy. The method used in ALSFR S-R score process of this six-month compassionate use of enteric capsules was the same as that used in six-month clinical trials for Radicava. The total change of ALSFRS-R scores for patients in Table 1 is (−10) and the mean decrease of six is (−1.67). The mean decrease in six-month clinical trial for edaravone administration (i.v.) was (−5.01). Using the same calculation method, the efficacy of the new drug capsule vs Edaravone was {5.01−1.67}/5.01=66.7%. More than 25% is considered to have a clear efficacy. It is worth noting that clinical trials on Radicava for all stages of ALS patients failed, referred to the Wikipedia website (Edaravone-Wikipedia). Meanwhile, the enteric capsules of DPL-002 can intervene in all stages of ALS, shown in Table 1.

### General Clinical Observations

No adverse effects were observed in duration.

### CONCLUSIONS

Regarding the efficacy of six-month duration, the aspirin-mimetic compound DPL-002 vs Edaravone showed 66.7% more effective in respect of ALSFRS-R score change, which remarkably surpassed Edaravone. In addition, capsules of PL-002 could intervene in all stages of ALS, better than Edaravone (oral or i.v.) for early ALS only. Especially for patients number 2 (early stage) and number 4 (middle stage), the change of each ALSFRS-R scores was zero, which meant no deterioration for the period of six months; the ALSFRS-R score change in patient number 5 (advanced stage) was 1, which meant to slightly reverse the progression of ALS. All of these data suggested that DPL-002 presently be the best medication for ALS patients and meet the unmet medical needs for ALS treatment.

It will be understood by those of skill in the art that the various embodiments and examples of the present disclosure are illustrative only and are not intended to limit the scope of the present disclosure, and numerous modifications can be made without departing from the spirit and scope of the invention disclosed and are intended to be encompassed by the present disclosure. All patent or non-patent literature cited are incorporated herein by reference in their entireties without admission of them as prior art.

What is claimed is:

1. A method of treating amyotrophic lateral sclerosis (ALS), comprising administering to a subject in need of treatment a therapeutically effective amount of an active pharmaceutical ingredient (API) selected from: 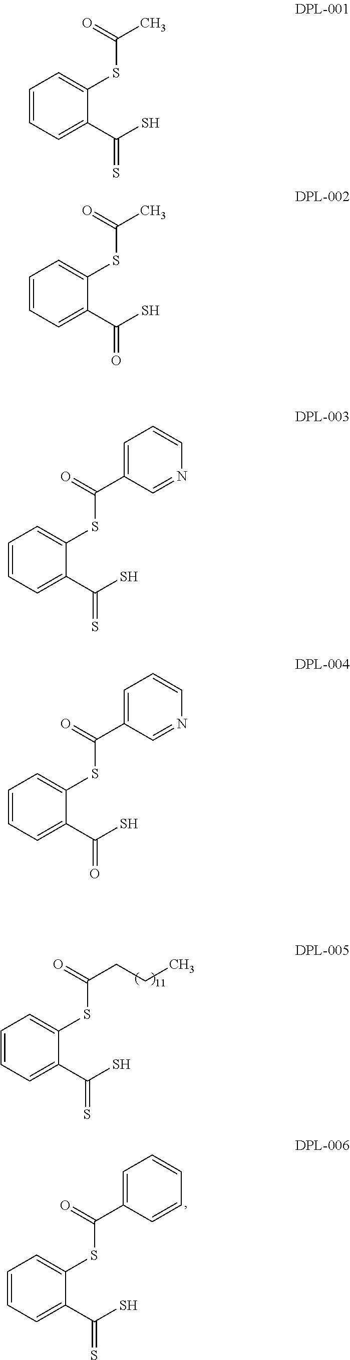
or a pharmaceutically acceptable salt thereof.

2. The method of claim 1, wherein the active pharmaceutical ingredient is DPL-002 or DPL-004.

3. The method of claim 1, wherein the active pharmaceutical ingredient is DPL-002.

4. An intravenous (i.v.) drip solution comprising an API compound selected from DPL-001, DPL-002, DPL-003, DPL-004, DPL-005, and DPL-006 according to claim 1, a pharmaceutically acceptable carrier, and optionally one or more pharmaceutical excipients.

5. The i.v. drip solution of claim 4, wherein the API compound is DPL-002; the pharmaceutically acceptable carrier is water; and the i.v. drip solution further comprises a pharmaceutical excipient independently selected from sodium chloride, potassium chloride, sodium hydrosulfite, and combinations thereof.

6. The i.v. drip solution of claim 5, wherein concentration of the API in the i.v. drip solution is in a range from about 0.1 mg/mL to about 30 mg/mL.

7. The i.v. drip solution of claim 5, wherein concentration of the API is in a range from about 1 mg/mnL to about 10 mg/mL.

8. The i.v. drip solution of claim 5, having a pH in the range from 6 to 8, inclusive.

9. An enteric capsule comprising an API compound selected from DPL-001, DPL-002, DPL-003, DPL-004, DPL-005, and DPL-006 according to claim 1, and optionally one or more pharmaceutical excipients.

10. The enteric capsule of claim 9, comprising a pharmaceutical excipient independently selected from starch powder, purified water, NaCl, KCl, dicalcium phosphate, magnesium stearate, carboxy methylcellulose sodium, and combinations thereof.

11. A method of treating ALS in a subject, comprising administering to the subject a therapeutically effective amount of the i.v. drip solution of claim 4.

12. The method of claim 11, wherein the API compound is DPL-002; and dose range of API in the i.v. drip solution administered to the subject is from about 5 mg to about 1500 mg daily, or from about 0.1 mg/kg body weight to about 30 mg/kg body weight.

13. The method of claim 11, wherein the API compound is DPL-002; and dose range of API in the i.v. drip solution administered to the subject is from about 0.5 μmol/kg body weight to about 150 μmol/kg body weight.

14. The method of claim 11, wherein the API compound is DPL-002; and the i.v. drip solution is administered to the subject in a frequency from one time daily to 5 times daily.

15. A method of treating ALS in a subject, comprising administering to the subject a therapeutically effective amount of the enteric capsule of claim 9.

16. The method of claim 15, wherein the API compound is DPL-002; and dose of the API in the enteric capsule is in a range from about 10 mg to about 3000 mg daily, or from about 0.2 mg/kg body weight to about 60 mg/kg body weight.

17. The method of claim 16, wherein the API compound is DPL-002; and dose of the API in the enteric capsule is in a range from about 1 μmol/kg body weight to about 300 μmol/kg body weight.

18. The method of claim 16, wherein the enteric capsule is administered to the subject in a frequency from 1 time daily to 5 times daily.
